(19) United States
(12) Patent Application Publication
(10) Pub. No.: US 2011/0202028 A1
(43) Pub. Date: Aug. 18, 2011

(54) RELEASE SHEET MATERIAL

(76) Inventors: Carlo Toro, Cepagatti, Pescara (IT); Norman BROYLES, Hamilton, OH (US); Enzo POMPEI, Pescara (IT); Fiorello SALONE, Pescara (IT); Arsen SIMONYAN, Montesilvano (IT); Andrew Julian WNUK, Wyoming, OH (US)

(21) Appl. No.: 13/023,943

(22) Filed: Feb. 9, 2011

Related U.S. Application Data
(60) Provisional application No. 61/304,898, filed on Feb. 16, 2010.

(51) Int. Cl. A61F 13/84; B32B 27/08; B32B 5/00; B29C 47/06; B29C 47/00; C08K 5/20

(52) U.S. Cl. 604/385.02; 428/515; 428/520; 428/476.3; 428/483; 428/516; 428/220; 428/336; 264/173.14; 264/176.1; 524/232

(57) ABSTRACT

Release sheet materials for use as packaging material for individually packaged disposable absorbent articles, typically sanitary napkins and the like.

### CROSS REFERENCE TO RELATED APPLICATIONS

This application claims the benefit of U.S. Provisional Application No. 61/304,898 filed on Feb. 16, 2010.

### FIELD OF THE INVENTION

The present invention relates to improved release sheet materials for use for example in connection with disposable absorbent articles, for example sanitary napkins and the like, which are typically individually packaged prior to use.

### BACKGROUND OF THE INVENTION

Disposable absorbent articles of personal hygiene are known in the art. Typical examples include sanitary napkins, panty liners, adult incontinence articles, infant diapers, paper towels, bath tissue and facial tissue. Such articles are often used to absorb and retain bodily fluids and other exudates excreted by the human body. Many disposable absorbent articles have the same basic structure: an absorbent core encased between a liquid permeable, user contacting topsheet, which permits liquid to penetrate its thickness and contact the absorbent core where liquid is retained, and a backsheet, which may be liquid impermeable.

While there are a great many variations in the specific structural features of disposable absorbent articles, they are typically presented to consumers in the same manner. Essentially, the disposable absorbent article, irrespective of the specific structural features used, is packaged in a box or bag from which the consumer withdraws the article, as needed. In order to protect the article from soiling or contamination from the time it is removed from the box or bag until the article is used, for example if a woman wanted to carry a sanitary napkin with her for use away from home, the articles may be individually packaged within the box or bag by means of a sheet of material which is wrapped around the individual article.

A typical individual package for disposable absorbent articles is disclosed, for example, in U.S. Pat. No. 4,556,146, which describes a disposable absorbent article, typically a sanitary napkin, associated with a wrapper which overlays one major surface of the sanitary napkin. The wrapper extends beyond the perimeter of the disposable absorbent article so that when the disposable absorbent article and the wrapper are folded as a unit, the longitudinal side flaps of the wrapper, which extend beyond the longitudinal sides of the article, may be frangibly sealed thereby providing the disposable absorbent article with an individual package, it is also common to provide the disposable absorbent article with an adhesive element on the garment facing side of the backsheet which, in use, serves to affix the absorbent article to the wearer's undergarment thereby maintaining the absorbent article in place against the wearer's body. The adhesive element may take the form of a coating of adhesive which is for example in strips or other suitable pattern. For example, the garment facing side of the backsheet can be coated uniformly with a layer of pressure sensitive hot melt adhesive. The wrapper overlays the garment facing side of the backsheet with the longitudinal flap portions extending beyond the longitudinal perimeter segments of the absorbent article. The wrapper typically is not folded onto or otherwise brought into contact with the body facing side of the topsheet; in other words, the surface of the wrapper facing the garment facing side of the backsheet is in face to face relation substantially with said side only of the backsheet. The wrapper is typically releasably affixed to the disposable absorbent article, e.g. a sanitary napkin, by the aforementioned adhesive element. When an adhesive element is used in this manner, it is not necessary to provide the absorbent articles with a separate release sheet in order to protect the adhesive element before use, as this function is provided by the wrapper.

To individually package the absorbent article, the article and the affixed wrapper can be typically folded as a unit, as described for example in the above mentioned U.S. Pat. No. 4,556,146. That is, they are folded together with the wrapper remaining in place with respect to the absorbent article. Typically, the absorbent article is folded lengthwise into thirds about two fold axes. The longitudinal side flaps or flap portions of the wrapper are frangibly sealed using any of the well known sealing techniques. For example, the longitudinal flap portions may be heat sealed, glued, ultrasonically bonded, or crimped.

In use, the individually packaged absorbent article is provided to a user. The user may then break the frangible seals, unfold the wrapper/absorbent article unit and separate the wrapper from the absorbent article, for example a sanitary napkin, exposing the adhesive element. The absorbent article may then be used as such devices normally are, typically being adhered by means of the adhesive element to the crotch portion of an undergarment, which is subsequently worn.

An advantage of an individually packaged disposable absorbent article, for example a sanitary napkin or a pantiliner, is discreetness, as a user does not need to take with her an entire box or bag of products, but only a small number of individually packaged articles, as needed for subsequent use, for example when staying away from home. In a high speed production line the material of the wrapper can be typically provided in a continuous web; the adhesive element may be provided in selected areas directly onto the wrapper material continuous web, and then absorbent articles are provided onto each respective adhesive element, by adhering thereon the garment facing side of the backsheet. The wrapper material continuous web may be folded together with the applied absorbent article, and cut and sealed in order to form individually packaged articles. When the user, after unfolding the package and breaking the seals, separates the absorbent article from the wrapper material, at least some of the adhesive element, and typically substantially the entire adhesive element, remains on the garment facing surface of the backsheet since the respective surface of the wrapper element is typically provided with means having an inferior adhesion to the adhesive material. Said means can be typically a release composition provided to the wrapper material for example as a coating.

The wrapper material, e.g. a polyolefin film, can include a silicone composition which provides the wrapper material with the needed inferior adhesion to the adhesive material, i.e., namely with its adhesive release property. The silicone composition can be typically provided to the already formed sheet material as a coating, for example a known coating technique comprises applying to the sheet material used for the wrapper a layer of a silicone precursor composition, for example comprising a crosslinkable organosilicon prepolymer, which is then cured, namely crosslinked, and anchored to the sheet material surface. Curing/crosslinking can be typically performed by providing energy to the precursor composition in presence of a suitable catalyst or initiator, for example as UV radiation in the presence of a photoinitiator, or heat in the presence of a catalyst.

Other release coating compositions as those disclosed in U.S. Pat. No. 2,880,862 can for example comprise a solution in organic solvent of an oil-modified alkyd resin with an amino-aldehyde release agent which can be for example ethylene bis-stearamide. Said solutions can be typically provided as a coating to the sheet material, for example a synthetic film, and are subsequently cured by heating in an oven in presence of a catalyst.

Providing the release composition, for example a silicone composition, as a coating to a sheet material, for example the wrapper material as described above, constitutes a multi step process, involving the formation, or in any case the provision, of a finished sheet material which has to be then coated with the release composition, e.g. a silicone precursor composition, followed by the curing/crosslinking step. Also, this process is energy consuming, as curing/crosslinking is achieved by providing energy.

Alternative processes for the production of wrapper materials, e.g. typically polyolefin films, already comprising a release material, e.g. a silicone based composition, have been developed in order to comprise a single manufacturing step, i.e. a film formation process, typically by extrusion, already including the release material.

A release sheet material, for example for a wrapper material, could in principle be obtained by directly extruding a layer from a masterbatch containing polyethylene and ethylene bis-stearamide as the release compound providing the release properties, or also by co-extruding such a layer together with at least another additional polyolefin layer, for example a polyethylene layer.

Said structures, however, are not satisfactory in terms of stability. In use, in fact, the ethylene bis-stearamide compound tends to at least partially migrate typically into the adhesive material in contact with it; for example the adhesive material of an absorbent article contacting it in the wrapped configuration as described above. This can spoil the adhesive properties of the adhesive material, which can hence show a lower adhesion capacity towards the fabric material of the undergarment. More in general, the ethylene bis-stearamide compounded with a polyolefin such as polyethylene has also a tendency to migrate into adjacent materials, e.g. other polyolefin co-extruded layers, possibly due to affinity with them, hence modifying the nature of said layers during time. This can in fact cause for example a decrease of the ethylene bis-stearamide content in the release sheet material, and ultimately a reduction of the release properties thereof.

It is therefore desirable to provide a flexible release sheet material for use for example as wrapper material for disposable absorbent articles, which comprises a single layer or a multi layer structure obtainable by means of extrusion or co-extrusion, and which is also more stable, i.e. less prone to the risk of material contamination, i.e. comprising a release material, typically a fatty acid amide material, for example ethylene bis-stearamide, which has a reduced tendency to migrate into adjacent layers and/or in the adhesive material in contact thereto, for example when used as a wrapper material for an individually wrapped absorbent article in the packaged configuration.

A flexible release sheet material can also find use in other applications different from wrapper materials for disposable absorbent articles, where release properties have to be shown towards adhesive or more in general sticky materials meant to come in contact therewith, for example as wrapper or lining material for adhesive tapes or sheets, or also rubber bales, food such as dried fruit, confections, baked goods and so on.

### SUMMARY OF THE INVENTION

The present invention addresses the above need by providing a flexible release sheet material comprising an extruded or co-extruded film comprising at least a first layer comprising a polyolefin or blend of polyolefins, from 1% to 5% by weight of a fatty acid amide or blend of fatty acid amides, and from 1% to 20% by weight of a compatibilizer compound selected from ethylene-acrylic ester-maleic anhydride terpolymers, ethylene-acrylic acid copolymers, ethylene-vinyl acetate copolymers, and polydimethylsiloxane-polyamide copolymers.

### BRIEF DESCRIPTION OF THE DRAWINGS

FIG. 1 is a partially cut away perspective view of an absorbent article and its associated wrapper prior to being folded and sealed.

FIG. 2 is a partially cut away perspective view of an absorbent article and its associated wrapper after they have been folded and sealed to form an individually packaged absorbent article.

FIG. 3 is cross section of a flexible release sheet material according to an embodiment of the present invention.

FIG. 4 is cross section of a flexible release sheet material according to another embodiment of the present invention.

FIG. 5 is a cross section of a flexible release sheet material according to a further alternative embodiment of the present invention.

FIG. 6 is a schematic representation of an apparatus for the manufacturing of a flexible release sheet material according to the present invention.

### DETAILED DESCRIPTION OF THE INVENTION

The present invention relates to a flexible release sheet material which may find use as a lining or wrapper material meant to come in contact with adhesive or sticky materials, and showing a reduced adhesion towards them. As a non limiting example, the flexible release sheet material of the present invention will be herein described in its use as a wrapper material for an individually packaged disposable absorbent article, wherein the article is adhered to the wrapper material through the adhesive which is meant, in use, for adhesion of the absorbent article to the user's undergarment, but can be extended to alternative executions where the adhesive in the absorbent article is for direct adhesion of the article itself to the user's body. More in general, the flexible release sheet material of the present invention may also find use in other different applications, where for example the release properties have to be applied to adhesive or more in general sticky materials meant to come in contact with the release sheet itself, for example as wrapper or lining material for adhesive tapes or sheets, or also rubber bales, food such as dried fruit, confections, baked goods and so on. Hence all respective teachings and features of the present invention as described herein are meant to apply to the flexible release sheet material as such independently of its intended use.

Referring now to the drawings there is shown an individually packaged disposable absorbent article illustrating the teachings of the present invention in an exemplary, but non limiting embodiment. As used herein the term “absorbent article” refers to those articles intended to absorb and retain liquid and in particular to those articles which are placed against or in proximity to a wearer's body to absorb and contain the various liquids discharged from the body (e.g. blood, menses, urine). A “disposable absorbent article” is an absorbent article which is intended to be discarded after a single use (i.e., it is not intended to be laundered or otherwise restored and reused). Exemplary disposable absorbent articles according to the present invention can be absorbent articles for feminine hygiene such as sanitary napkins and panty liners, commonly referred to collectively as catamenial pads, light incontinence products, or the like.

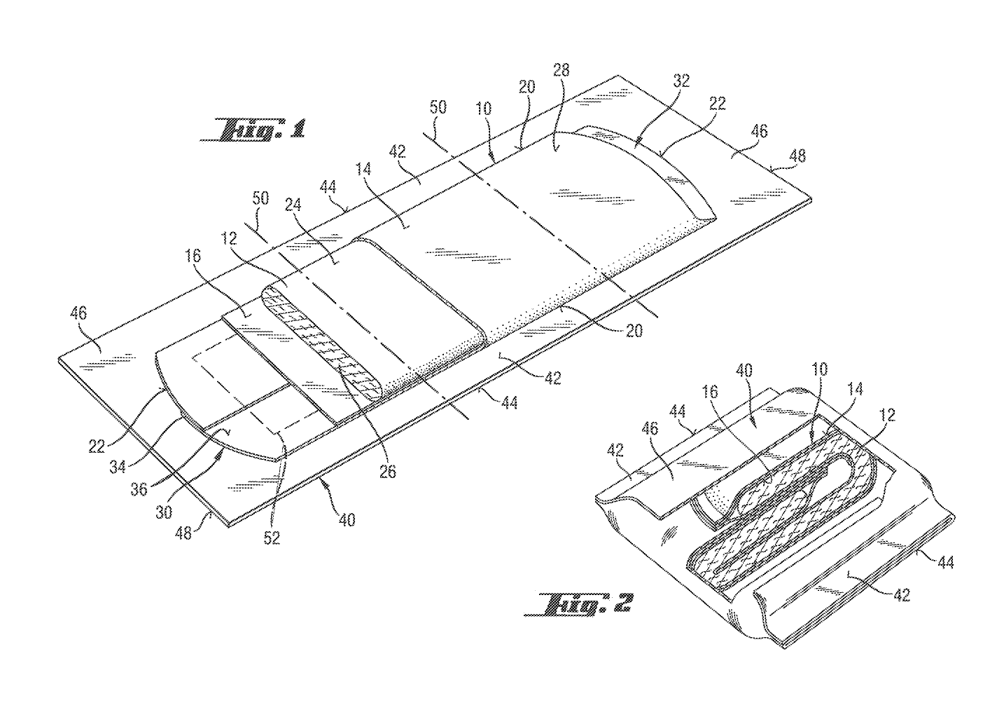

FIG. 1 is a partially cut away perspective view of a catamenial pad 10 and a wrapper 40 prior to being folded and sealed as set forth in greater detail herein below. A catamenial pad is a disposable absorbent article which is worn by females external to and in the proximity of the urogenital region and which is intended to absorb and contain menstrual fluids and other vaginal discharges. As used herein the term “catamenial pad” includes pantiliners which are often worn by females external to the urogenital region between periods of heavy menstrual flow and which are intended to absorb light menstrual flow and nonmenstrual vaginal discharges. The primary difference between catamenial pads used during periods of heavy menstrual flow and catamenial pads used between periods of heavy menstrual flow (i.e., pantiliners) is the absorbent capacity of the pad.

While the present invention will be described with reference to a catamenial pad, it should be understood that the present invention has application in the context of other disposable absorbent articles such as, for example, light incontinence products, as well as outside of the context of disposable absorbent articles. Further, the teachings of this invention have application to catamenial pads manufactured according to the teachings of any of the multitudinous references in the catamenial pad art. A typical catamenial pad embodiment will now be described briefly.

As can be seen in FIG. 1, the catamenial pad 10 basically can comprise an absorbent pad 12, an envelope sheet 14 and a barrier 16. The catamenial pad 10 has a perimeter generally comprising longitudinal perimeter segments, or longitudinal sides 20 and transverse perimeter segments, or transverse ends 22. The perimeter defines the outer boundary of the catamenial pad 10, while the longitudinal perimeter segments 20 and the transverse perimeter segments 22 define the outer boundary of the catamenial pad 10 along each longitudinal side and each transverse end respectively.

The absorbent pad 12 is generally compressible, conformable, and non-irritating to the user's skin and may be manufactured from a wide variety of absorbent materials which are capable of absorbing and retaining liquids. For example, a batt of absorbent fibres, a multiplicity of plies of creped cellulose wadding, or any equivalent material may be used. The absorbent capacity of the material used, however, should be sufficient to absorb and retain the expected liquid loading in the intended use of the absorbent article without undue bulk. An example of a suitable catamenial pad 10 intended to receive heavy menstrual discharges of approximately 40 millilitres, may include about 8 grams of comminuted wood pulp, generally referred to as airfelt.

The shape and dimensions of the absorbent pad 12 can be selected to permit the disposable absorbent article to conform to and fit about the portion of the body against which it will be placed. Often, as in the embodiment illustrated, the general shape and dimensions of the catamenial pad 10 can be determined by the shape and dimensions of the absorbent pad 12. In the embodiment illustrated in the figures, the shape and dimensions of the absorbent pad 12 were selected to permit the catamenial pad 10 to conform to the urogenital region of the wearer's body. While the shape and dimensions of the absorbent pad 12 may be varied, it has been found that a generally planar configuration having a first major surface, or body facing side, 24 and a second major surface, or garment facing side, 26 can be suitable. The first major surface 24 is that surface of the absorbent pad 12 facing toward the source of liquid (i.e. toward the wearer's body) and the second major surface 26 is that surface of the absorbent pad 12 facing away from the source of liquid. An example of a suitable catamenial pad 10 can have a generally rectangular, planar shaped absorbent pad 12 having a length of about 9.0 inches (22.9 centimetres) and a width of about 2.5 inches (6.4 centimetres). It should be understood, however, that other shapes (e.g. elongated ovals, triangles, squares, etc.) and other dimensions may be used.

The catamenial pad 10 may include an envelope sheet 14 that encases the absorbent pad 12 and is preferably compliant, soft feeling, and non-irritating to the wearer's body. The envelope sheet 14 can help maintain the structural integrity of the absorbent pad 12 and has a first and a second end flap 30 and 32 respectively. The envelope sheet 14 may be wrapped about the absorbent pad 12 and may be affixed to itself along a seam 34 which is adjacent the second major surface 26 and which traverses the catamenial pad 10 longitudinally. The first and second flaps 30 and 32 respectively extend beyond the transverse ends of the absorbent pad 12 and are typically sealed so as to completely encase the absorbent pad 12 within the envelope sheet 14. The portion of the envelope sheet 14 overlaying the first major surface 24 is the topsheet portion 28 corresponding to the body facing side of the article, and the portion of the envelope sheet 14 overlaying the second major surface 26 is the backsheet portion 36 of the envelope sheet 14, corresponding to the garment facing side of the article. The topsheet portion 28 is liquid permeable. In use the topsheet portion 28 may contact the skin of the catamenial pad wearer and permit the transmission of liquid through its thickness to the absorbent pad 12 where the liquid may be retained.

There are many suitable materials from which the envelope sheet 14 may be manufactured. The topsheet portion 28 may be manufactured from either hydrophobic or hydrophilic fibres and may, for example, be carded, spun bonded, melt blown, or air laid. Alternatively, the topsheet portion 28 may be a continuous film or sheet of, for example, thermoplastic material which is apertured. A suitable topsheet portion 28 is described in U.S. Pat. No. 4,324,246 which issued to Mullane et al on Apr. 13, 1982.

The topsheet portion 28 and the backsheet portion 36 may either be integral (i.e. the backsheet portion 36 and the topsheet portion 28 are separate elements affixed to each other) or unitary (i.e. the backsheet portion 36 and the topsheet portion 28 are formed from a continuous and undivided sheet of material) and may either have the same or different physical properties. The embodiment of FIG. 1 shows the topsheet portion 28 and the backsheet portion 36 as being unitary.

To help prevent liquids absorbed by the absorbent pad 12 from penetrating through the backsheet portion 36, it may be advantageous to interpose a barrier 16 at the interface between the second major surface 26 of the absorbent pad 12 and the backsheet portion 36. The barrier 16 may be manufactured from any flexible, liquid impermeable material which is non-irritating to the wearer. In certain embodiments, the barrier 16 may be a sheet of polyethylene which is coincident with the backsheet portion 36.

Alternatively, the envelope sheet 14 may comprise a topsheet portion 28 and a backsheet portion 36 which are made integral with each other by affixing them together about their periphery. In such embodiments, the topsheet portion 28 may be at least partially liquid impervious and the backsheet portion 36 may be liquid pervious or wholly or partially liquid impervious.

It is common to provide the catamenial pad 10 with an adhesive element 52, partially shown in dotted line in FIG. 1. The adhesive element 52 is positioned on the garment facing side of the article, namely on the backsheet portion 36 and, in use, serves to affix the catamenial pad 10 to the wearer's undergarments thereby maintaining the catamenial pad 10 in place against the wearer's body. The adhesive element 52 may take the form of a coating of adhesive which is in strips or any other suitable pattern. The backsheet portion 36 may be coated uniformly with a layer of a pressure sensitive hot melt adhesive such as, for example, NS34-2823 as manufactured by National Starch and Chemical of Bridgewater, N.J.

In accordance with the teachings of the exemplary embodiment of this invention, a wrapper 40 is associated with, and typically can have dimensions generally larger than those of the catamenial pad 10. Thus, the wrapper 40 has longitudinal flap portions 42 comprising that portion of the wrapper 40 between the longitudinal edge 44 of the wrapper and the longitudinal perimeter segment or longitudinal side 20 of the catamenial pad 10. In the embodiment illustrated in FIG. 1, the wrapper 40 also has transverse flap portions 46 comprising that portion of the wrapper 40 between the transverse edges 48 of the wrapper 40 and the transverse perimeter segments or transverse ends 22 of the catamenial pad 10.

The wrapper 40 is shown overlaying the garment facing side of the absorbent article, namely the backsheet portion 36, with the longitudinal flap portions 42 typically extending beyond the longitudinal perimeter segments 20, in the absorbent shown in FIG. 1, the wrapper 40 is not folded onto or otherwise brought into contact with the topsheet portion 28. In other words, the surface of the wrapper 40 facing the backsheet portion 36 is in face to face relation with the backsheet portion 36 only. The wrapper 40 is releasably affixed to the catamenial pad 10 by the aforementioned adhesive element 52, hence it is also defined as a releasable wrapper 40 according to the present invention. If an adhesive element is used in this manner, it is not necessary to provide the absorbent article with a separate release sheet as is commonly done in prior art devices, as the function of protecting the adhesive element from contamination prior to use is provided by the wrapper.

To individually package the catamenial pad 10, the catamenial pad 10 and the affixed wrapper 40 can be folded as a unit. That is, they can be folded together with the wrapper 40 remaining in place with respect to the catamenial pad 10. According to the exemplary embodiment of the present invention, the absorbent article and the wrapper can be folded as a unit about at least one fold-axis, e.g. a longitudinal or a transverse fold-axis, in order to define a packaged absorbent article. In certain embodiments, the catamenial pad 10 may be folded lengthwise into thirds about two transverse fold-axes 50, as shown in FIG. 2, to define a package comprising the absorbent article. The longitudinal flap portions 42 can be frangibly sealed using any of the well-known sealing techniques. For example, the longitudinal flap portions 42 may be heat sealed, glued, or ultrasonically bonded.

In use, the individually packaged catamenial pad is provided to a user. The user may then break the seals, unfold the catamenial pad 10, and separate the wrapper 40 from the catamenial pad 10. The catamenial pad 10 may then be used as such devices normally are.

Means having a reduced adhesion to the adhesive material of the adhesive element 52 can be typically provided into the material of the wrapper 40, namely on the surface which is meant to contact, and be affixed to, the adhesive element 52 on the garment facing surface of the absorbent article in an individually packaged and wrapped absorbent article as illustrated in FIGS. 1 and 2.

According to the present invention, as illustrated in FIG. 3, a flexible release sheet material is provided, typically for use as a releasable wrapper 40 for an individually packaged absorbent article. The flexible release sheet material comprises the film 55, typically obtained by extrusion, which can be for example a single layer film. The flexible release sheet material comprises a polyolefin or blend of polyolefins, from 1% to 5% by weight, or from 1.5% to 4% by weight, or also from 2% to 3% by weight of a fatty acid amide, or blend of fatty acid amides, and from 1% to 20% by weight, or from 2% to 15% by weight, or also from 4% to 13% by weight, of a compatibilizer compound selected from ethylene-acrylic ester-maleic anhydride terpolymers, ethylene-acrylic acid copolymers, ethylene-vinyl acetate copolymers, and polydimethylsiloxane-polyamide copolymers, and mixtures thereof.

The polyolefin or blend of polyolefins can comprise polyethylene, or polypropylene, or blends or copolymers thereof. Typically the polyolefin can be polyethylene, for example high density polyethylene (HDPE), or low density polyethylene (LDPE), or linear low density polyethylene (LLDPE), or a blend of LDPE and LLDPE, such as for example a blend of four parts LLDPE and one part LDPE.

It is believed the adhesive release property in the flexible release sheet material of the present invention is provided by the presence in the material itself of a fatty acid amid; or blend of fatty acid amides.

Fatty acid amides can be selected among amides of a saturated or unsaturated carboxylic acid, for example a monocarboxylic acid, or alkylene bis-amides of a saturated or unsaturated monocarboxylic acid. The amides of a saturated or unsaturated carboxylic acid can be selected among primary amides of carboxylic acids having from 12 to 22 carbon atoms. Thus, for example, there may be used amides of lauric, myristic, palmitic and stearic acids as well as diamides of dicarboxylic acids such as oxalic and adipic acids and amides of unsaturated acids such as oleic and erucic acids. The fatty acid amides according to the present invention can also be selected among alkylene bis-amides which have the structure:

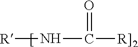

wherein R′ represents an alkylene group having from 1 to 5 carbon atoms and R represents an alkyl group of from 12 to 22 carbon atoms. For example, typical alkylene bis-amides can be selected among ethylene, methylene and propylene bis-amides, such as for example methylene bis-stearamide, ethylene bis-stearamide, ethylene bis-palmamide, propylene bis-stearamide; propylene bis-oleamide, and the like. For example, the fatty acid amide or blend of fatty acid amides according to the present invention can be selected among methylene bis-stearamide, ethylene bis-stearamide, and propylene bis-stearamide. Typically, the fatty acid amide can be ethylene bis-stearamide.

According to the present invention, the compatibilizer compound can be typically selected among ethylene-acrylic ester-maleic anhydride terpolymers, ethylene-acrylic acid copolymers, ethylene-vinyl acetate copolymers, and polydimethylsiloxane-polyamide (PDMS-PA) copolymers, and mixtures thereof.

According to the present invention the compatibilizer compound can be for example an ethylene-acrylic ester-maleic anhydride terpolymer. Typically, ethylene-acrylic ester-maleic anhydride terpolymers can have a relatively low maleic anhydride content, for example below 1% by weight, down to about 0.3% by weight, while the acrylic ester content can be from 10% to 30% by weight. The compatibilizer compound can be for example selected among the ethylene-acrylic ester-maleic anhydride terpolymers sold by Arkema under the trade name Lotader® MAH, such as for example Lotader® 4403.

The compatibilizer compound can be also an ethylene-acrylic acid copolymer. Typically the content of acrylic acid can be for example from 1% to 25% by weight, or also from 5% to 15% by weight. An example can be the compound sold as Primacor® 3460 by Dow Chemical, from the Primacor® family.

The compatibilizer compound can be also an ethylene-vinyl acetate copolymer. Typically the content of vinyl acetate can range for example from 9% to 25% by weight. An example can be a compound selected from the Elvax® resins sold by DuPont and having the preferred vinyl acetate content.

The compatibilizer compound can be also selected among polydimethylsiloxane-polyamide (PDMS-PA) copolymers. Suitable PDMS-PA copolymers can have at least one polydimethylsiloxane block of individual molecular weight between 1.000 Da and 100.000 Da, or between 3.000 Da and 30.000 Da, or also between 5.000 Da and 15.000 Da, and at least one polyamide block of individual molecular weight between 1.000 Da and 10000 Da, or between 1000 Da and 50.000 Da, or also between 4.000 Da and 20.000 Da. The overall molecular weight of the PDMS-PA copolymer can be between 2.000 Da and 2.000.000 Da. The PDMS-PA copolymer can typically have a melt flow index (M.F.I.), evaluated by the ASTM method D 1238-85 under conditions 190/2.16, of between 0.5/10 min and 300 g/10 min, or between 1 g/10 min and 100 g/10 min, or also between 1 g/10 min and 30 g/10 min. The PDMS-PA copolymer can also have a melting point between 80° C. and 200° C., where, as it is known for polymers, the melting point is the DSC (Differential Scanning calorimetry) melting point, which is the temperature identified as that corresponding to the DSC peak, or corresponding to the highest DSC peak in case of a mixture of polymers showing more than one peak. The PDMS-PA copolymer can also have the polyamide selected among 4-6, 6-6,6,6-9, 6-11, or 6-12 types, typically a 6-11, 6-12 or a 6-6 polyamide.

According to the present invention, the compatibilizer compound can be typically a polydimethylsiloxane-polyamide copolymer. Typically, the polydimethylsiloxane-polyamide copolymer can be selected in order to have one or more of the characteristics described above. Without being bound to any theory, it is believed polydimethylsiloxane-polyamide copolymers are particularly suitable as compatibilizer compounds towards fatty acid amides in the flexible release sheet materials of the present invention, based on their respective chemistry, since both compounds comprise amide groups in the molecule. It has also been found that polydimethylsiloxane-polyamide copolymers selected as a compatibilizer compound in the formulation of the flexible release sheet material according to the present invention can have the further advantage of providing a sheet material producing a reduced noise upon separation of the adhesive material therefrom; for example, in the exemplary embodiment illustrated in FIG. 1, when the absorbent article is detached from the release sheet material of the wrapper the noise is low, thus making the article more discreet in its current use.

The flexible release sheet material of the present invention can also comprise a co-extruded multilayer film comprising at least two layers, wherein at least one first layer consists of a film comprising a polyolefin or blend of polyolefins, a fatty acid amide or blend of fatty acid amides and a compatibilizer compound, with the components selected as described above. The co-extruded multilayer film also comprises a second layer comprising a polyolefin or blend of polyolefins, for example the second layer can actually consist of a polyolefin or blend of polyolefins. This provides for example a flexible release sheet material of the desired thickness, which comprises a thicker layer made of a cheaper material, e.g. typically a polyolefin, as the second layer described above, and only having at least one external relatively thin layer of the composition meant to provide the release properties on at least one side of the multilayer film structure, actually the first layer described above.

Such a multilayer film can be typically made by means of co-extrusion, which refers to the extrusion of multiple layers of material simultaneously. This type of extrusion can typically utilize two or more extruders to melt and deliver a steady volumetric throughput of different viscous thermoplastic materials to a single extrusion head (die) which will extrude the materials in the desired form, i.e., as a multilayer film. This technology can be used on any of the processes for film forming, such as sheet/film extrusion or blown film extrusion. The respective layer thicknesses can be controlled according to known means, i.e. typically by the relative speeds and sizes of the individual extruders delivering the materials to the single extrusion die. Co-extrusion can have the advantage that a multiple layer film structure can be manufactured in a single and relatively simple process step, with no need of post processing. For example a composite film can be produced having a layer or layers providing mechanical characteristics such as flexibility, resilience, strength, and a layer or layers providing desired surface finishing, such as for example an adhesive release surface.

According to the present invention, a flexible release sheet material comprising a co-extruded multilayer film, i.e. a multilayer film obtainable by means of co-extrusion, can hence be provided which has both mechanical and chemical stability, for example for use in a releasable wrapper for an individually packaged absorbent article. A releasable wrapper made of this release sheet material, and an absorbent article individually packaged in a releasable wrapper made of this flexible release sheet material are also within the scope of the present invention. The flexible release sheet material, as e.g. a co-extruded multilayer film, can be obtained by means of a careful selection of the components of the different layers.

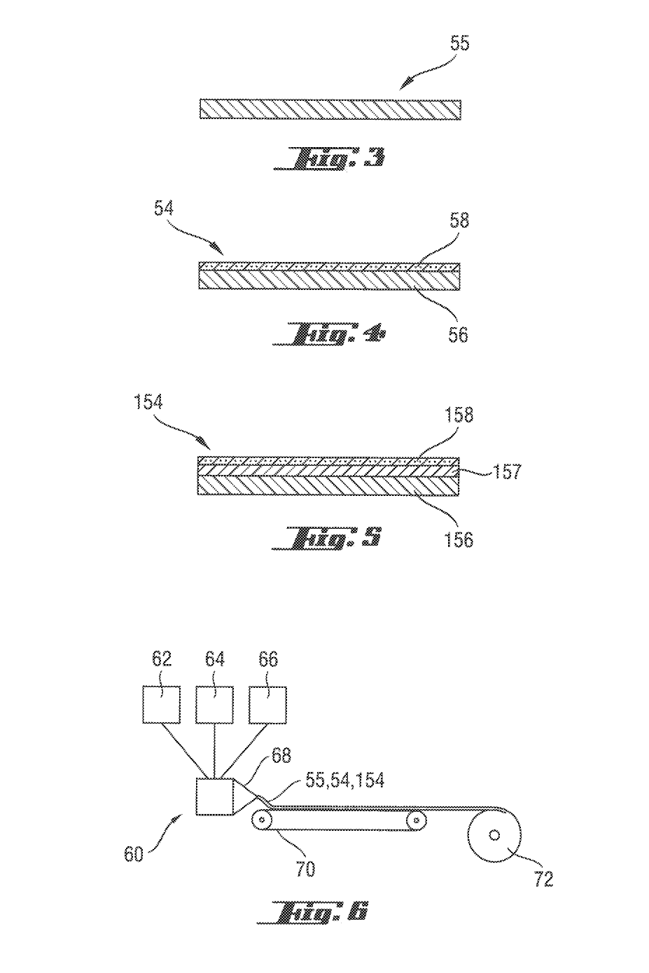

As schematically illustrated in FIG. 4, the flexible release sheet material according to this embodiment of the present invention comprises the co-extruded multilayer film 54, which can typically include at least two layers, respectively at least a first layer 58 comprising a polyolefin or blend of polyolefins, from 1% to 5% by weight, or from 1.5% to 4% by weight, or also from 2% to 3% by weight of a fatty acid amide or blend of fatty acid amides, and from 1% to 20% by weight, or from 2% to 15% by weight, or also from 4% to 13% by weight, of a compatibilizer compound selected from ethylene-acrylic ester-maleic anhydride terpolymers, ethylene-acrylic acid copolymers, ethylene-vinyl acetate copolymers, and polydimethylsiloxane-polyamide copolymers, or mixtures thereof, and a second layer 56 comprising a polyolefin or blend of polyolefins. The composition and the components of the first layer 58 can be typically selected as described above with reference to the single layer film 55 of FIG. 3, and can typically correspond thereto. Typically, the second layer 56 can actually consist of a polyolefin or blend of polyolefins, selected as mentioned above, i.e. comprising polyethylene, or polypropylene, or blends of copolymers thereof. Typically the polyolefin can be polyethylene, for example low density polyethylene (LDPE), or linear low density polyethylene (LLDPE), or a blend of the two, such as for example a blend of four parts LLDPE and one part LDPE.

According to another embodiment of the present invention, a flexible release sheet material can comprise a co-extruded multilayer film 154 as illustrated in FIG. 5, comprising a first layer 158 and a second layer 156 typically having the same compositions as corresponding first and second layers 58 and 56 described above with reference to FIG. 4; the second layer 156 can further comprise a polyamide or polyethylene terephthalate. Typically, the second layer 156 can consist of a polyolefin or blend of polyolefins, for example of polyethylene, selected as mentioned above with respect to the second layer 56. The coextruded multilayer film 154 further comprises a third layer 157 between the first layer 158 and the second layer 156. The third layer 157 can be typically constituted of a compatibilizer compound selected from ethylene-acrylic ester-maleic anhydride terpolymers, ethylene-acrylic acid copolymers, ethylene-vinyl acetate copolymers, and mixtures thereof. Typically the compatibilizer compound of the third layer 157 can be selected in order to correspond to the compatibilizer compound comprised in the first layer 158, unless the compatibilizer compound of the first layer 158 is a PDMS-PA copolymer, as it has been discovered that a PDMS-PA copolymer is not suitable as an intermediate third layer 157 in a coextruded multilayer film 154 as illustrated in FIG. 5. An intermediate third layer 157 constituted by the compatibilizer compound selected as said above, and interposed between the first and the second layers 158, 156, can further improve the bonding between the second layer 156 comprising the polyolefin or blend of polyolefins, and the first layer 158 providing the release properties.

According to an embodiment of the present invention the flexible release sheet material can be actually constituted by a co-extruded multilayer film as described above with reference to FIGS. 4 and 5.

The multilayer construction of the co-extruded flexible release sheet materials 54, 154 of the present invention, as represented for example by the embodiments of FIGS. 4 and 5, generally at least comprises a second layer 56, 156 typically constituted by a polyolefin, or a blend of polyolefins, and typically providing the multilayer release sheet material 154 with the desired mechanical properties, and at least a first layer 58, 158 typically comprising a polyolefin or blend of polyolefins, a fatty acid amide or blend of fatty acid amides, and a compatibilizer compound, selected as described above, which instead can be typically meant to provide the release properties. This provides the advantage of having a multilayer release sheet material mostly comprising inexpensive but mechanically resistant polyolefin or polyolefins, and at least only a thin layer made of the composition which provides the release properties.

Without being bound to any theory, it is believed the presence of the selected compatibilizer compound in the composition comprising the polyolefin or polyolefins and the selected fatty acid amide or blend of fatty acid amides, either constituting the single layer flexible release sheet material 55, illustrated for example in FIG. 3, or a first layer 58, 158 in a multilayer flexible release sheet material, such as sheet materials 54 and 154 illustrated in FIGS. 4 and 5 respectively, chemically stabilizes the fatty acid amide or amides within the polyolefin, and makes it less prone to migrate in other materials which contact the sheet material itself, such as for example an adhesive, when the release sheet material is typically used as a release material intended to releasably contact an adhesive, for example in the context of a release wrapper material for an individually packaged absorbent article, as illustrated in FIGS. 1 and 2. The improved stability of the fatty acid amide or amides within the polyolefin can be also beneficial in the context of a multilayer release sheet material, such as in the embodiment illustrated in FIG. 4, where possible migration of the fatty acid amide from the first layer 58 towards the second layer 56 is prevented. This effect is further reinforced in the embodiment illustrated in FIG. 5, where the second layer 156 is separated from the first layer 158 containing the fatty acid amide or amides by a further intermediate layer 157 typically constituted by the compatibilizer compound only, which acts as a sort of “barrier” layer.

According to the present invention, the flexible release sheet material can be formed, as a single layer or as a multilayer film, as exemplified for example in the embodiments of FIGS. 3, 4 and 5 respectively, in the desired thickness by extruding the above composition in conventional extrusion or co-extrusion apparatuses, typically as sheet/film or blown film extrusion or co-extrusion. Typical thicknesses for the flexible release sheet material of the present invention may vary according to the final use of the material itself, and can range from 8 μm to 80 or even up to 100 μm, or from 10 μm to 40 μm, or also from 15 μm to 30 μm. These ranges may be typical for use of the flexible release sheet material as a wrapper material for an individually packaged disposable absorbent article.

In release sheet materials having multilayer film structures, such as those illustrated in FIGS. 4 and 5, the thickness of the second layer 56, 156 comprising the polyolefin or blend of polyolefins, can be from 5 μm to 30 μm, or from 8 μm top 20 μm, or also from 10 μm to 15 μm. The thickness of the first layer 58, 158, comprising the polyolefin or blend of polyolefins, the fatty acid amide or blend of fatty acid amides, and the compatibilizer compound, can be from 0.5 μm to 5 μm, or from 1 μm to 3 μm. The thickness of the third intermediate layer 157, when present, as shown in FIG. 5, between the first layer 158 and the second layer 156, and consisting of a compatibilizer compound, can be from 1 μm to 10 μm, or from 4 μm to 8 μm.

A process and an apparatus for making a flexible release sheet material according to the present invention is schematically illustrated, in a simplified way, in FIG. 6; the apparatus shown can relate to the extrusion of a single layer film 55, as that illustrated in FIG. 3, as well as to the co-extrusion of a multi layer flexible sheet material, for example a two or three layer as shown in FIGS. 4 and 5, being appropriately adaptable as known in the art. An apparatus 60 for extrusion or co-extrusion is fed through reservoirs 62, 64, 66 of the selected polyolefin material, of the selected fatty acid amide or amides, and of the selected compatibilizer compound, respectively. The different materials can be mixed together as appropriate with known means, not shown, and provide the selected feed or feeds to the extrusion die 68 in order to form a single layer film 55 or a multilayer film 54, 154 having the desired structure, i.e. comprising for example two or three layers with specific compositions as explained above. The film can be moved through e.g. a conveyor belt 70 to further manufacturing steps, or wound in a roll 72 for storage and subsequent use.

The flexible release sheet material according to the present invention comprising an extruded or co-extruded film can be produced with a simpler and cost effective process, typically consisting of a single manufacturing step. The flexible release sheet material of the present invention can also comprise further components, such for example inks, dyes, opacifiers, in order to modify the appearance, e.g. providing a colour, of the finished film; such components can be typically added for example directly to the appropriate feed of the extrusion apparatus.

The flexible release sheet materials of the present invention are also more stable and resistant, particularly those comprising co-extruded multilayer structures, which are less prone to e.g. delamination when subjected to subsequent mechanical treatments, such as for example embossing.

The invention will be illustrated by the following examples, where by PE/EBS masterbatch it is meant a homogeneous blend of polyethylene and 5% by weight ethylene bis-stearamide, for example available as RAC-0500 masterbatch from Polyfil Corp. (Rockaway, N.J.). All percentages are by weight, unless otherwise stated.

### Example 1

A flexible release sheet material according to the invention was prepared by extruding a single layer film 25 μm thick comprising 60% PE/EBS masterbatch, 28.8% of a polyolefin blend of 4 parts LLDPE and 1 part LDPE and 11.2% of a polydimethylsiloxane-polyamide compatibilizer compound sold by Dow Corning under the code DC 2-8179.

### Example 2

A flexible release sheet material according to the invention was prepared by extruding a single layer film 25 μm thick comprising 30% PE/EBS masterbatch, 65% of a polyolefin blend of 4 parts LLDPE and 1 part LDPE and 5% of a polydimethylsiloxane-polyamide compatibilizer compound sold by Dow Corning under the code DC 2-8179.

### Example 3

A flexible release sheet material according to the invention was prepared by extruding a single layer film 25 μm thick comprising 30% PE/EBS masterbatch, 60% of a polyolefin blend of 4 parts LLDPE and 1 part LDPE, 5% of a polydimethylsiloxane-polyamide compatibilizer compound sold by Dow Corning under the code DC2-8179 and 5% of an ethylene-acrylic ester-maleic anhydride terpolymer compatibilizer compound sold by Arkema as Lotader® 4403.

### Comparative Example

A sheet material was prepared by extruding a layer film 25 μm thick comprising 50% PE/EBS masterbatch and 50% of a polyolefin blend of 4 parts LLDPE and 1 part LDPE.

### Reference

As a reference, a commercial release sheet material sold by Nordenia under the code NOR® Pouch RPW PE NDG V5 was also provided, consisting of a LDPE film which has received a UV cured silicone coating according to the known technology,

### Release Force Test

The release force of the sheet materials was tested according to the FINAT Test Method no. 10, from the FINAT Technical Handbook 6^{th} edition, 2001, using a pressure sensitive adhesive tape Tesa® 4154 available from Tesa Tape Inc., Charlotte, N.J., USA. The test measures the force in Newton necessary to detach the standard pressure sensitive adhesive tape which has been adhered to the release sheet material. The test method has been slightly modified as for the Test Conditions. Accelerated ageing is selected for the test strips, but they are kept for 20 hours in an air circulating oven at 40° C.±3° C., instead of 70° C.±5° C. as prescribed in the test method.

### Adhesive Contamination Test

The adhesive contamination is expressed in terms of the amount of fatty acid amide, namely ethylene bis-stearamide in the Examples described above, migrated into the adhesive tape after it has been detached from the release layer in the test for the release force. This can be suitably qualitatively evaluated by means of Fourier Transform Infra Red Spectroscopy (FTIR). The tape strips are taken from the Release Force Test and immediately after subjected to FTIR spectroscopy on the adhesive side; the area of the tape strips actually examined is in the centre of each tape strip, namely the centre of the area of the adhesive previously adhered to the release sheet. The samples are analyzed using a Perkin Elmer Spectrum One FTIR spectrometer equipped with the Universal Diamond ATR (Attenuated Total Reflectance) accessory. Spectra are made up of 16 scans with a resolution of 4 cm⁻¹ ranged between 4000 cm⁻¹ and 650 cm⁻¹; to verify the changes in the spectra, the N—H stretching of the amide at about 3300 cm⁻¹ is used; the wavelength range actually considered is from 3500 cm⁻¹ to 3100 cm⁻¹. The amount of amide migrated into the adhesive, in turn representative of the adhesive contamination, is proportional to the peak height of the amide, expressed in units of “relative absorbance”, and directly obtainable from the instrument software. As known with results, the values of peak height reported in the table do not represent as such any actual amount of amide within the adhesive; they are instead proportional to the amount of amide migrated into the adhesive, and can be used to make comparisons among the different examples. Any other suitable method and equipment for FTIR can be also used in order to qualitatively evaluate the presence of the fatty acid amide in the adhesive material of the adhesive tape, since the comparison of the results from the different samples has to be taken into account, while the actual values obtained typically do not correspond to any specific amount for the fatty acid amide which has migrated.

The results are summarized in the table below.

| | | | | | | | Adhesive |
|---|---|---|---|---|---|---|---|
| | Layer | Composition of film in wt. % | | | | Release | contamination |
| | Thickness | PE/EBS | | Lotader ® | LDPE/ | force | (relative |
| Example | (μm) | masterbatch | PDMS-PA | 4403 | LLDPE | (N) | absorbance) |
| 1 | 25 | 60 | 11.2 | — | 28.8 | 0.1 | 0 |
| 2 | 25 | 30 | 5 | — | 65 | 0.6 | 0 |
| 3 | 25 | 30 | 5 | 5 | 60 | 1 | 0.0030 |
| Comp. | 25 | 50 | — | — | 50 | 2.4 | 0.0043 |
| Ref. | 25 | n/a | n/a | n/a | n/a | 0.2 | 0 |

The results in the table show that the comparative example not only provides a rather high release force, typically not acceptable in the context of a releasable wrapper for an individually packaged absorbent article, as evidenced by the comparison with the Reference, but also causes contamination of the adhesive, which in use translates in a reduced adhesive capacity of the adhesive material after contact with the release material for a given time. In the field of individually packaged disposable absorbent articles, this corresponds to a reduced capacity of the absorbent article of adhering to the user's undergarment after being detached from the wrapper material.

The examples of release sheet material made according to the invention instead combine a low release force, slightly higher than that of the Reference material for Examples 2 and 3, but still acceptable, and even lower for Example 1, moreover virtually eliminating contamination of the adhesive, as shown by the negligible presence of ethylene bis-stearamide found within the adhesive; no contamination at all is actually shown for Examples 1 and 2. Also, the flexible release sheet material according to the present invention is made according to a simple and cost effective process in a single manufacturing step, moreover using cheaper raw materials if compared to e.g. the Reference material, constituted by a polyolefin film provided with a UV cured silicone based release coating.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A flexible release sheet material comprising an extruded or co-extruded film comprising at least a first layer comprising:
a polyolefin or blend of polyolefins,
from 1% to 5% by weight, preferably from 1.5% to 4% by weight, more preferably from 2% to 3% by weight of a fatty acid amide or blend of fatty acid amides, from 1% to 20% by weight, preferably from 2% to 15% by weight, more preferably from 4% to 13% by weight, of a compatibilizer compound selected from ethylene-acrylic ester-maleic anhydride terpolymers, ethylene-acrylic acid copolymers, ethylene-vinyl acetate copolymers, and polydimethylsiloxane-polyamide (PDMS-PA) copolymers, and mixtures thereof.

2. A flexible release sheet material according to claim 1, wherein said material is a co-extruded film and further comprises a second layer comprising a polyolefin or blend of polyolefins.

3. A flexible release sheet material according to claim 2, wherein said second layer can further comprise a polyamide or polyethylene terephthalate, said sheet material further comprising a third layer comprised between said first layer and said second layer, said third layer constituted of a compatibilizer compound selected from ethylene-acrylic ester-maleic anhydride terpolymers, ethylene-acrylic acid copolymers, ethylene-vinyl acetate copolymers, and mixtures thereof.

4. A flexible release sheet material according to claim 3, wherein said fatty acid amide or blend of fatty acid amides is selected among amides of a saturated or unsaturated carboxylic acid, and alkylene bis-amides of a saturated or unsaturated monocarboxylic acid.

5. A flexible release sheet material according to claim 4, wherein said fatty acid amide or blend of fatty acid amides is selected among methylene bis-stearamide, ethylene bis-stearamide, and propylene bis-stearamide.

6. A flexible release sheet material according to claim 5, wherein said fatty acid amide is ethylene bis-stearamide.

7. A flexible release sheet material according to claim 1, wherein said compatibilizer compound is an ethylene-acrylic ester-maleic anhydride terpolymer or a polydimethylsiloxane-polyamide (PDMS-PA) copolymer.

8. A flexible release, sheet material according to claim 7, wherein said compatibilizer compound is polydimethylsiloxane-polyamide (PDMS-PA) copolymer.

9. A flexible release sheet material according to claim 2, wherein said polyolefin is polyethylene.

10. A flexible release sheet material according to claim 9, wherein said second layer is constituted by polyethylene.

11. A flexible release sheet material according to claim 1, wherein said material has a thickness of 8 μm to 80 μm, preferably of 10 μm to 40 μm, more preferably of 15 μm to 30 μm.

12. A flexible release sheet material according to claim 1, wherein said first layer has a thickness of 0.5 μm to 5 μm, preferably from 1 μm and 3 μm.

13. A flexible release sheet material according to claim 1, wherein said second layer has a thickness of 5 μm to 30 μm, preferably from 8 μm and 20 μm, more preferably from 10 μm and 15 μm.

14. A flexible release sheet material according to any of claim 1, wherein said third layer has a thickness of 1 μm to 10 μm, preferably from 4 μm and 8 μm.

15. A releasable wrapper for an individually packaged absorbent article, said releasable wrapper comprising a flexible release sheet material according to any preceding claim.

16. An individually packaged absorbent article comprising an absorbent article having a body facing side, a garment facing side, two longitudinal sides and two transverse ends, said absorbent article having an adhesive element on said garment facing side, a releasable wrapper overlaying said garment facing side of said article and releasably affixed to said adhesive element, said absorbent article and said wrapper being folded as a unit about at least one fold-axis to define a package comprising said absorbent article, wherein said releasable wrapper is according to claim 15.

17. A process for making a flexible release sheet material according to claim 1, comprising the steps of:
providing at least a first feed of a first material comprising a polyolefin or blend of polyolefins, from 1% to 5% by weight, preferably from 1.5% to 4% by weight, more preferably from 2% to 3% by weight of a fatty acid amide or blend of fatty acid amides, and from 1% to 20% by weight, preferably from 2% to 15% by weight, more preferably from 4% to 13% by weight, of a compatibilizer compound selected from ethylene-acrylic ester-maleic anhydride terpolymers, ethylene-acrylic acid copolymers, ethylene-vinyl acetate copolymers, and polydimethylsiloxane-polyamide (PDMS-PA) copolymer, and mixtures thereof;
optionally providing a second feed of a second material comprising a polyolefin or blend of polyolefins;
optionally providing a third feed of a third material comprising a compatibilizer compound selected from ethylene-acrylic ester-maleic anhydride terpolymers, ethylene-acrylic acid copolymers, ethylene-vinyl acetate copolymers, and mixtures thereof;
extruding or co-extruding said first material, optionally said second material and optionally said third material into a single layer or multi layer film, wherein said first material provides a first layer, said optional second material provides an optional second layer, and said optional third material provides a further optional third layer comprised between said first layer and said optional second layer.
